(19) United States
(12) Patent Application Publication
(10) Pub. No.: US 2019/0095579 A1
(43) Pub. Date: Mar. 28, 2019

(54) BIOMECHANICAL MODEL GENERATION FOR HUMAN OR ANIMAL TORSI

(71) Applicant: KONINKLIJKE PHILIPS N.V., EINDHOVEN (NL)

(72) Inventors: Dominik Benjamin Kutra, Karlsruhe (DE); Thomas Buelow, Grosshansdorf (DE)

(21) Appl. No.: 16/086,376

(22) PCT Filed: Mar. 20, 2017
(86) PCT No.: PCT/EP2017/056483

§ 371 Date: Sep. 19, 2018

(30) Foreign Application Priority Data
Mar. 31, 2016 (EP) 16163126.2

(51) Int. Cl. G06T 7/00; G06F 17/50; A61F 2/12

(52) U.S. Cl. CPC G06F 19/26; G06T 7/0014; A61F 2/12; G06T 2200/04; G06T 2207/30068; G06F 17/5009

(57) ABSTRACT

System and related method of generating a composite model for a bio-mechanical assembly. The comprises an input interface (IN) for receiving i) at least two input component models (m(B), m(C)) for respective anatomical components (B, C) of the mechanical assembly, and ii) a surface image acquired by a camera (DSC) of an outer layer (OL) of said biomechanical assembly (T). A combiner (Σ) is configured to combine, based on said surface image, said at least two input component models (m(B), m(C)) into a combined mechanical model (m(T)) for said biomechanical assembly.

### FIELD OF THE INVENTION

The invention relates to a system of generating a composite model for a bio-mechanical assembly, to a method of generating a composite model for a bio-mechanical assembly, to a computer program element and to a computer readable medium.

### BACKGROUND OF THE INVENTION

Breast cancer is the most common cancer type that inflicts women in the western world.

Patients have several treatment options, e.g. surgery, where the affected part of the breast tissue is removed. In the context of joint decision making, biomechanical methods have been developed to simulate the outcome of breast surgery in advance. The simulation provides the patient or medical staff with a visual representation of the surgical outcome to better understand the consequences.

Presently, the production of such simulations of biomechanical assemblies (such as the human torso) is based on MR or CT images acquired for any one of a specific patient and models constructed from such imagery. Said differently, before the simulation for a given patient anatomy can be computed, imagery of the internals of the specific patient needs to be acquired first. However, acquisition of such imagery is either very expensive or constitutes in itself health risks due to radiation dosage in case of X-radiation.

### SUMMARY OF THE INVENTION

There may therefore be a need for alternative systems or methods to facilitate or make safer the production of models for simulation of biomechanical assemblies.

The object of the present invention is solved by the subject matter of the independent claims where further embodiments are incorporated in the dependent claims. It should be noted that the following described aspect of the invention equally applies to the method of generating a composite model, to the computer program element and to the computer readable medium.

According to a first aspect of the invention there is provided a system of generating a composite model for a bio-mechanical assembly, comprising:

an input interface for receiving i) at least two input component models for respective anatomical components of the mechanical assembly, and ii) a surface image acquired by a camera of an outer layer of said biomechanical assembly; and

a combiner configured to combine, based on said surface image, said at least two input component models into a combined mechanical model for said biomechanical assembly.

In one embodiment, the model components are 3D or volumetric model components, one of the components is situated behind the other, and both behind the outer layer (eg skin), relative to the camera. The at least two anatomical components are as such visually occluded from the camera. The camera preferably uses non-ionizing radiation for the surface imaging. At least locally, a shape of the outer layer conforms at least partly with the shape of at least a part of at least one of the anatomical components. The components are coupled to each other and/or at least one of them is coupled to the outer layer. Yet more specifically and according to one embodiment, the bio-mechanical assembly is an animal or human torso and the anatomical components include a) a breast and b) at least a part of the chest wall.

Generating a composite super-model from two or more sub-models as proposed herein allows capturing a wider range of anatomical variations and thus achieving more realistic biomechanical simulations as opposed to monolithic model constructions that aim to model as one whole the components in the biomechanical assembly.

According to one embodiment, at least one of said at least two input component models previously adapted from a generic model for a respective one of the internal components.

According to one embodiment, the at least two input models have been so adapted from respective generic models, with said generic models separately learned from respective, different, training sets. This allows avoiding use of in particular MRI imagery thus saving time and cost for preparing the simulation.

According to one aspect, the combiner includes a solver component configured to:

join a first one of the input component models to a second one of the input component models at an initial position of said second input component model, to so obtain a candidate combined mechanical model;

perform a mechanical simulation of the candidate combined model to obtain a first configuration of the candidate combined model;

compare said configuration of the candidate combined model with the surface image to obtain a measure of deviation; and

based on said measure of deviation, to vary said initial position to obtain a second candidate combined model.

According to one embodiment, the bio-mechanical simulation is performed over a plurality of mechanical degrees of freedom (DoFs). The DoFs include rotation, translation and/or deformation. A higher level of realism is achievable.

According to one embodiment, said varying of said initial position is performed only for a subset of said plurality of said mechanical degrees of freedom. This allows reducing computation time.

According to one embodiment, the solver proceeds iteratively in iteration steps through a series of candidate combined models, wherein a number of the mechanical degrees of freedom varies with said iteration steps. This allows reducing computation time.

According to another aspect there is provided a computer-implemented method of generating a composite model for a biomechanical assembly, comprising the steps of:

receiving i) at least two input component models for respective anatomical components of the mechanical assembly, and ii) a surface image acquired by a camera of an outer layer of said biomechanical assembly; and

based on said surface image, combining said at least two input component models into a combined mechanical model for said biomechanical assembly.

According to one embodiment, at least one of said at least two input component models previously adapted from a generic model for a respective one of the internal components.

According to one embodiment, the at least two input models have been so adapted from respective generic models, with said generic models separately learned from respective, different, training sets.

According to one embodiment, the combining step comprises the following sub-steps:

from the at least two input component models, joining a first one of the input component models to a second one of the input component model at an initial position of said second input component model, to so obtain a candidate combined mechanical model;

performing a mechanical simulation of the candidate combined model to obtain a first configuration of the candidate combined model;

comparing said configuration of the candidate combined model with the surface image to obtain a measure of deviation; and

based on said measure of deviation, varying said initial position to obtain a second candidate combined model.

According to one embodiment, the mechanical simulation is performed over a plurality of mechanical degrees of freedom.

According to one embodiment, said varying of said initial position is performed only for a subset of said plurality of said mechanical degrees of freedom.

According to one embodiment, the method proceeds iteratively in iteration steps through a series of candidate combined models, wherein a number of the mechanical degrees of freedom varies with said iteration steps.

According to one embodiment, the mechanical assembly is an animal or human torso.

According to one embodiment, the internal components include a) a breast and b) a chest wall.

According to another aspect, there is provided a computer program element, which, when being executed by a processing unit, is adapted to perform the method of any one of above mentioned embodiments.

According to yet another aspect there is provided a computer readable medium having stored thereon the program element.

In short, and according to one embodiment, a system and related method is proposed that enables the generation of models for biomechanical simulation of in particular the human torso without the need for MR or CT imaging, purely based on a representation of the surface of the torso which can be gained in one preferred embodiment from optical surface scans.

### BRIEF DESCRIPTION OF THE DRAWINGS

Exemplary embodiments of the invention will now be described with reference to the following drawings (which are not necessarily to scale) wherein:

FIG. 1 is a schematic drawing of anatomical components of a mechanical bio-medical assembly;

FIG. 2 shows a schematic block diagram of a system for generating a composite model for a mechanical bio-medical assembly;

FIG. 3 shows a flow chart of a method of generating generic input models of anatomical components of a bio-mechanical assembly;

FIGS. 4A, B show flow charts of a method of generating a composite model for a bio-mechanical assembly; and

FIG. 5 shows illustrations of a method of registering a surface image to a model.

### DETAILED DESCRIPTION OF EMBODIMENTS

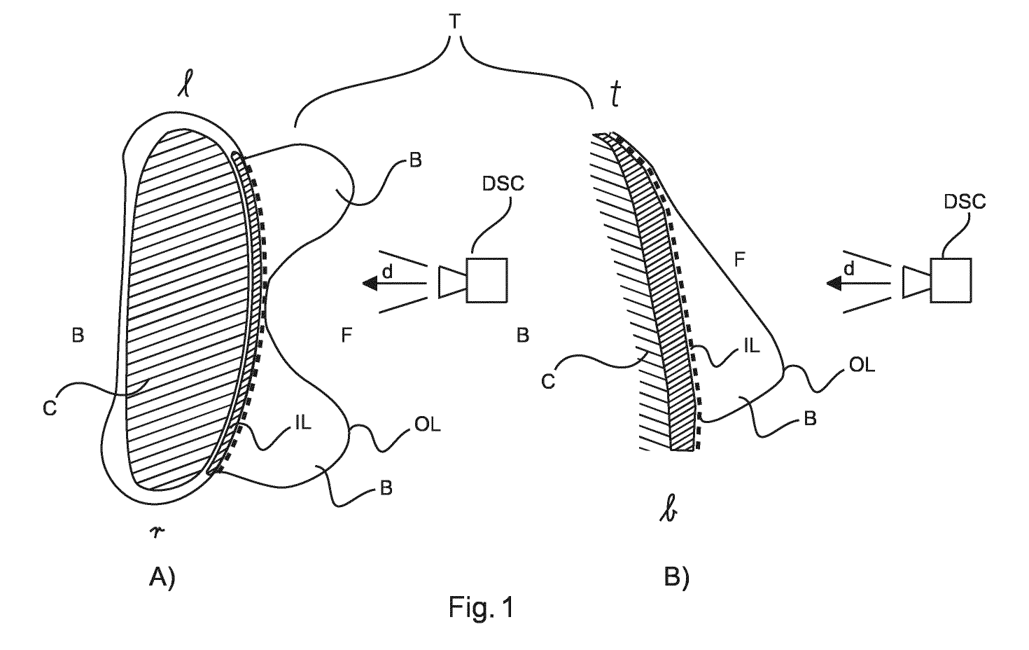

What is proposed herein is a computerized model generator system CMG for generating a composite model of a bio-mechanical assembly based on two or more input models. Each input model represents a respective anatomical part of the bio-mechanical assembly. Before explaining operation of the composite model generator system CMG in more detail it may be beneficial to refer first to FIG. 1 below (which is not necessarily to scale) to illustrate an exemplary bio-mechanical assembly.

FIG. 1A shows an axial view of a biomechanical assembly, such as a female human torso TOR. FIG. 1B on the other hand affords a sagittal view (side elevation) of said torso TOR. In FIGS. 1A, B, the reference character l and r indicate left and right, respectively, whereas reference characters t and b refer to, respectively, top and bottom portions of torso TOR. Structurally speaking, the torso TOR has a layered composition, one layer arranged behind the other, with the exposed outer layer OL, the skin that is, occluding the layers within the torso TOR. The two breasts are formed as bulged portions of the skin OL caused by the underlying breast tissue bulging into the skin OL. The breast tissue couples to a portion of the chest wall C within the torso. It is in particular this portion which is referred to herein as the chest wall element C. More particularly, the breast tissue is coupled to the rib cage wall element. The wall element C is a mix of a bone (ribs) and pectoral muscle tissue. There is also a further, intermediate or inner layer IL which is situated partly in between skin/breast tissue and the wall element and/or is partly surrounding (that is, is adjacent to) the breast tissue. This inner layer IL is formed from connective tissue, but also breast tissue and fat tissue. The main components of the female torso looked at as a biomechanical assembly includes thus the outer layer (skin), the underlying breast tissue, the chest wall C which forms a wall to which the breast tissue is coupled to via the intermediate layer situated between breast tissue and the rib cage. The dashed line in FIG. 1 shows the particular shape of the chest wall C.

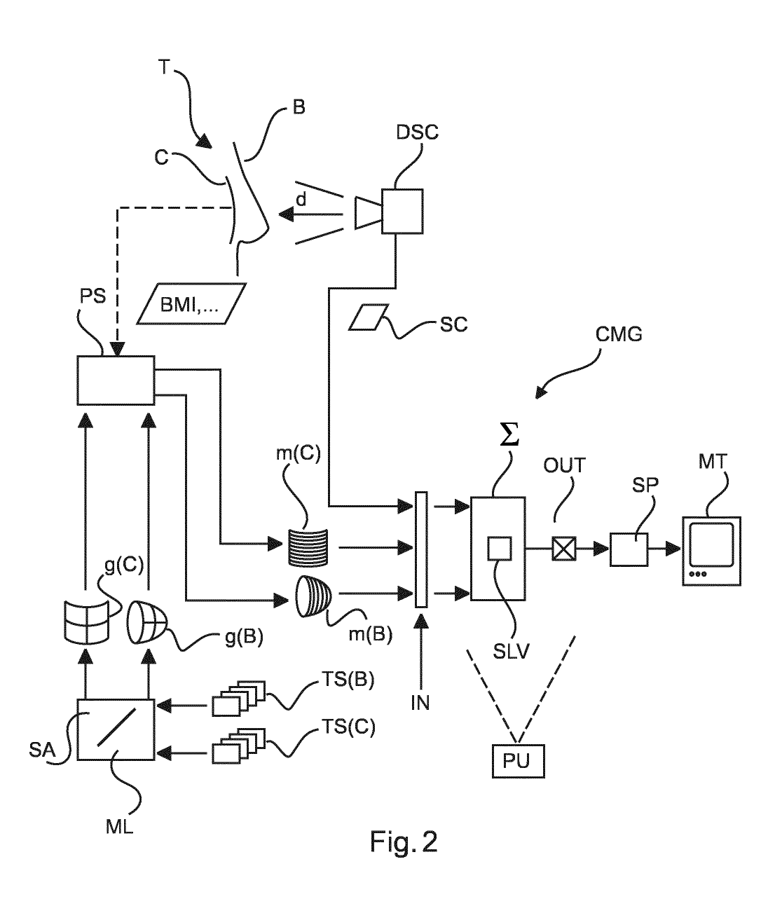

Reference is now made to FIG. 2 where a schematic block diagram of the proposed composite model generator system CMG (also referred to herein simply as the “composite model generator”) is shown. The system optionally includes the surface camera DSC (for instance, a Microsoft Kinect camera), although this is not strictly necessary as the surface imagery SC can also be retrieved as earlier acquired imagery from a picture or image storage system rather than being supplied directly by the camera DSC. Other optical techniques such as stereo-imaging, laser scanner systems (with or without time-of-flight) are also envisaged as are non-optical techniques such as echolocation or others, although optical systems are preferred because of their higher accuracy.

The processing components of the composite model generator CMG system can be implemented as software modules on a general purpose computing unit PU such as in surgical planning workstation. In alternate embodiments, the modules of the IPS in FIG. 2 are arranged in a distributed architecture and connected in a suitable communication network. The modules may be arranged in hardware as suitably programed FPGAs (field-programmable gate array) or as hardwired integrated circuits. The system may also be implemented as an “app” for various tablet or smartphone platforms such as google glass, Android™ or iOS tablets, or others.

Although operation of the composite model generator CMG will be explained herein with main reference to the torso T as described above in FIG. 1, this is not necessarily limiting, as application of the proposed composite model generator CMG to other bio-mechanical assemblies in humans or animals are also contemplated herein.

The input models m(C) and m(B) may be taken to represent respectively the chest wall C and one of the breast B. The method can be readily extended to both breasts with a composite model which includes chest wall and the two breasts, in which case the input is formed from three (or more) models.

The input models m(C) and m(B) as well as the generated composite model m(T) are each computerized representations of the respective anatomies and may be formed as mesh models. The models may include surface models or volume models. The models are formed from geometrical elements as known from FEA (finite element analysis) such as triangular or other geometrical elements. In case of volume models, the elements are formed as 3D geometrical elements such as tetrahedrons or others. The elements together defined the respective mesh model m(B), m(C) or m(T). The volume/surface elements are joined together at their edges/faces. Some or each of the elements can be labelled to encode anatomic information. Each element can be subjected to “virtual” mechanical force to simulate deformation, motion or other DoFs (degrees of freedom). To achieve more realistic (bio-) mechanical simulations, some or each element of the respective mesh is addressable to encode local material parameters (such as local elasticity parameters) to so define an overall dynamic behaviour under load that is characteristic for the particular anatomy (breast B, chest wall C, etc) to be modelled.

The composite model generator CMG composes, that is, essentially fuses, the two separate input models MC and MB into a single model assemblage, that is, the composite model MT. The composite model MT can then be subjected to a common bio-mechanical simulation to simulate for instance the appearance of the combined assembly m(T) under influence of a force field such as the gravity field or to simulate removal of matter from the torso to so simulate surgical procedures.

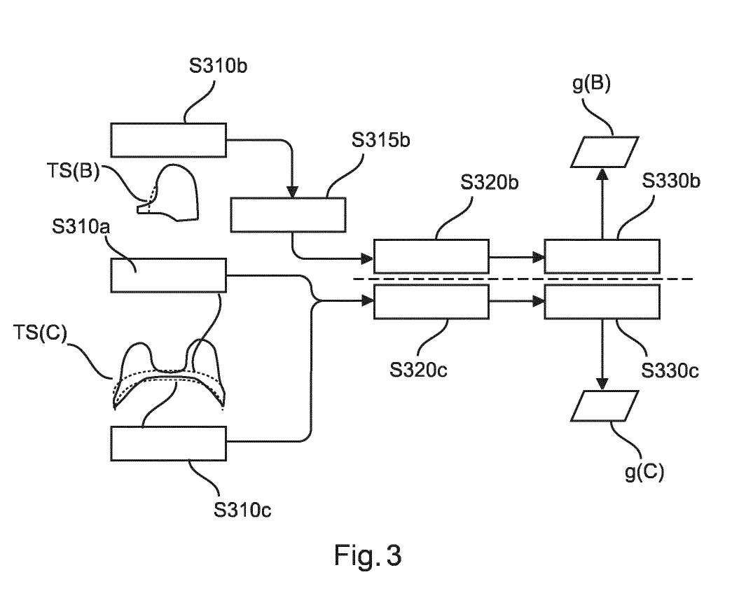

With continued reference to FIG. 2, the two (or more) input models m(C), m(B) for the breast B and the chest wall C, respectively) are received at input port IN. As a matter of principle, the composite model generator CMG can receive as input any type of input models, no matter how they have been generated. However, in one embodiment, the two input models are generated in a particular manner as will be detailed further below with reference to FIG. 3.

With continued reference to FIG. 2, a further input is formed by the surface image SC acquired by the camera DSC as explained above in FIG. 1. The (one or more) input surface image CS (or “scan”) was previously acquired along one or more imaging direction(s) d as shown in FIG. 1. The one or more surface images SC preferably encode spatial depth information of the skin OL. As such, the two anatomies, breast B and chest wall

C, are visually occluded from the camera's vision but the skin OL at least partly follows the contour of the breast tissue B and a part of the chest wall C lateral to where the breast couples into the chest wall C. The camera preferably uses non-ionizing radiation for the surface imaging.

Based on the surface image SC, a combiner Σ module of the generator CMG combines the two input models m(C) and m(B) into the composite model m(T) which is then output at output port OUT.

The composite model m(T) is stored in a suitable data structure such as pointer structure or otherwise. The output composite model MT can be stored in a storage system or can be otherwise processed.

For instance, as indicated earlier, the combined model m(T) can be passed to a bio-mechanical simulation package SP. A non-limiting example for such a package is the open source system “Nifty Sim” which is based on the total Lagrangian explicit dynamic solver algorithm TLED, reported by K Miller et al in “Total Lagrangian explicit dynamics finite element algorithm for computing soft tissue deformation”, in Commun. Numer. Meth. Engineering, vol 23, pp 121-134 (2007). However, other FEM (finite-element-method) packages may be used instead. Based on the user specified conditions (eg, the specification of motion equation, applicable force fields, such as gravity field, and the desired mechanical degrees of freedom which one wishes to simulate for), the simulation package SP then generates a graphical representation of the simulation (in particular as a moving picture) which can be rendered for display on a display device such as a monitor MT. A predefined equation of motion is integrated in time-increments, proceeding in steps from an initial configuration to a final configuration. In contrast to other integrators, the TLED algorithm integrates in each step from a fixed, initial configuration rather than from each of a series of intermediate configurations. Preferably, a Lagrangian formulation for the motion equation is used but other formulations are also envisaged. Instead of displaying the time evolution of the simulation, it may be desired to simply display only a still image of the final configuration at the conclusion of the time integration.

As will be explained in more detail below, the surface image and one or both of the input models MC and MB may represent the anatomies and the torso in different orientations relative to a force field, in particular relative to the gravitational field. For instance, the breast model may represent the breast anatomy in supine or prone (or in fact in a force field free (eg, gravity free) configuration) whereas the chest wall represents the same in an upright position similar to the upright position at which the surface scan SC has been recorded by camera DSC.

The proposed composite model generator CMG does not require that the two input models and/or the surface image SC have each been recorded in the same orientation relative to the gravitational field. Specifically, at least one of the three orientations may be different or in fact each of the orientations may be different relative to the specified force field.

However, before explaining the operation of the composite model generator CMG in more detail, reference is now made to FIG. 3 where a procedure is outlined for obtaining the input models m(B), m(C) for breast B and the chest wall C, respectively, with the understanding that the proposed system CMG is not necessarily reliant on this procedure but accepts input models generated in whichever way.

More specifically, and according to one embodiment, the instant input models m(B), m(C) are obtained indirectly from respective generic models g(B) and g(C) for the respective partial anatomies, breast B and chest wall C. More specifically, FIG. 3 flowchart shows a method for generating the respective (in this non-liming case two) generic models g(B) and g(C) for the two component anatomies breast B and chest wall C of torso T.

Yet more specifically, training procedures for the respective generic models g(B), g(C) are proposed and these training procedures are de-coupled from each other as indicated by the horizontal dashed line in FIG. 3. In other words, the breast properties and the properties of the torso/chest-wall are trained independently on respective, different, training sets TS(B) and TS(C) because shape, size and tissue composition of the breast do not necessarily correlate with the torso dimensions. In one embodiment, and as schematically illustrated to the left of FIG. 3, the training stets are formed from historical image data earlier acquired from a population of patients and/or their associated meta-data. The associated meta-data or descriptors include additional information (on top of the bare image) of the individuals in the population, such as age, BMI, menopausal status, ethnicity, etc.

In the example, the training set imagery is formed from prone breast MRI data, but other orientations and/or other modalities capable of soft-tissue contrast are also contemplated herein. It will be understood that the respective training sets for the breast and chest wall may be comprised in the same image (series) but different parts of the image will be considered separately in the two training procedures. In other embodiments, different sets of imagery are used for each anatomy. The training sets comprise advantageously 3D image data received from a population of the respective anatomy. The training stets can be obtained from hospital information system (HIS) or picture archive storage systems (PASC) as maintained by hospitals or other medical facilities.

It will be understood from the above that the generation of the two generic models is essentially a one-off operation. Only then there is reliance on “expensive” image material of the internal anatomies, such as, in particular, the MRI imagery. These generic models can then be personalized by personalizer module PS to obtain the above mentioned input models m(B) and m(C) for input into the composite model generator CMG. Specifically, no MRI imagery is required to construct the input model for a given/instant patient (torso T) for whom the personalized input models m(B),m(C) and, ultimately, the composite model m(T) is to be generated. By “personalization” we understand steps to obtain from the generic model, the specific input models m(C), m(B) adapted to the surface scan SC of the patient/torso T for which the composite model m(T) is to be ultimately generated by generator CMG.

The method for generating the generic models is essentially a multi-prong procedure due to the decoupling in respect of the component anatomies B,C. Turning first to the training prong for the generic breast model gB, at step S310B a plurality of mesh models are fitted to the multiple images (eg MRI) from the respective corpus TS(B). This mesh fitting step may include additional steps such as noise filtering, segmenting and labeling the (raw) image data (such as the 3D MRI image data) of the training set TS(B).

In case of the breast or other anatomies, there may be a material analyzer step S315B to analyze the tissue distribution based on the information encoded in the MRI images of the training set.

In one embodiment, a statistical approach is taken to extract the information from which the generic model g(B) is to be built. Specifically, at step S320B a statistical analysis is carried out by statistical analyzer SA to learn the probability distribution of the shape model parameters which depend on the geometric primitives used for the selected models. For instance, in one embodiment a semi-ellipsoidal model (or more generally an ellipsoidal cap) is used as the primitive form of the breast g(B), and the parameters then include the lengths of the principal axes of the ellipsoid. The statistical analysis may include estimating mean and/or variance (or higher moments) or other statistics to describe the distribution.

At step S330B the statistical shape data learned in step S320B is correlated with the meta-data in the respective set TS(B). In other words, the dependency between meta-data or measurements and shape parameters is established. For instance, a mean girth of the breast for women having a certain age range and BMI range can be obtained. Similar analysis is done for shape parameters other than girth. Instead of correlation with meta-data, available measurements such as the breast tissue composition as per step S315B or the pectoral-muscle-to-mammilla-distance (based on historic x-ray mammograms) or others may be correlated with the statistical shape information.

The output is the generic breast model g(B) which can then be personalized (on which further below) to the personal data of the specific torso for which the composite model m(T) is to be generated. In other words, shape parameters of the generic model g(B) can be personalized based on the values of meta-data or non-image data measured for the individual patient.

Instead of or in addition to the statistical analysis, a machine learning procedures ML is used to extract the information to build the generic model. In the earlier, statistical approach, the training sets are looked at as “samples”, drawn from a wider female population. In the machine learning approach, a slightly different view is taken. Specifically, the training sets TS(A), TS(B) are now understood to be instances of respective examples of the functional relationship between shape as encoded by the image data and the respective meta-data of the respective patient. Image data and associated meta-data for each individual from the training corpus TS(B) (or TS (C) for the case of the chest wall) define a training pair (meta-data versus shape). Each of those training pairs can then be fed into a machine learning algorithm to learn the functional relationship between meta-data and shape configurations. Suitable machine learning algorithms include decision trees, support vector machines, neural networks or other.

In case machine learning is used to learn the generalized models g(B), g(C) these encode the functional relationship between the respective shapes and the descriptor or meta-data which have been used in the training procedure. The generic model is a function ƒ that allows to compute for a given meta-data or measurement (collectively denoted md), a shape s f(md)=s. The function ƒ is in generally not a closed analytic description, but this function is implicitly encoded in a look-up table or in the specific configuration of parameters of the machine learning algorithm, which parameters have been adjusted during the training.

A similar procedure, de-coupled from the breast procedure steps A310A-S330A outlined above, is carried out in steps S310C through steps S330C to derive a generic model g(C) for the chest wall C. The geometric primitive for the chest wall C is in one embodiment a cylinder (in particular, at least a part of its lateral surface) with elliptic cross-section.

In addition to generating the two generic sub-models g(B) and g(C) for breast B and chest wall C, a respective generic model for the inner layer IL can also be learned as indicated by step S310A (the remaining steps are not shown as these are completely analogous to the steps S320A,B and S330A,B above). Alternatively, the mesh data for the inner layer obtained at step S310A can be merged with the mesh model learned for the chest wall at step S310C and these two mesh models can then be processed together at step S320C-S330C as shown in FIG. 3, the resulting generalized model for the chest then including information about the inner layer IL as a “sub-sub” component.

Modelling separately for the inner layer IL and including this as an additional anatomy component into the composite model m(T) is advantageous as this allows achieving a particularly realistic biomechanical simulation of the torso. The inner layer IL effectively surrounds or embeds that part of the breast tissue that connects to the chest wall C. Knowledge of the shape and/or in particular the thickness of this inner layer has been observed by applicant to lead to highly realistic biomechanical simulations. It has been found furthermore that a thickness estimation of the inner layer can be based on prior knowledge learned from a population of torsi similar to what has been explained above in relation to the breast model m(B) or chest model m(C).

Once the generic models gC, gB, and if required g(IL) are obtained as per FIG. 3 above, these are then personalized by a personalization component PS based on the specific meta-data of the instant torso T for which the composite model is to be generated. Again, the meta-data or descriptors include for instance the BMI (body mass index), age or other data or measurements of the instant patient for whom the composite torso model m(T) is to be generated.

In one embodiment, the personalizer PS may make use of the upright surface scan SC and may also use additional image material such as x-ray mammography and basic meta-data which can be fetched from the patient file or can be supplied by the clinician by a suitable user interface to the personalizer PS.

Specifically, and according to one embodiment, the personalization of the generic chest wall model g(C) can be obtained in a direct procedure through the SC surface scan as both, the surface scan SC and the training data (on which the generic chest model g(C) is based) have been acquired in the same orientation relative to the gravitational field. In particular, the model part of chest wall model g(C) that corresponds to the skin surface OL is directly fitted to the upright 3D scan SC of the patient. For estimation of the underlying chest wall shape, some of the meta-data of the patient such as BMI is used to estimate the distance from chest-wall C to skin OL. The information of this skin-chest wall distance can be gained from prior knowledge and statistical analysis or machine learning.

As to the personalization of the generic breast model g(B) to the instant torso T, this may not be necessarily done directly by geometrical fitting as the 3D surface scan SC and the model may relate to different orientations relative to the gravitational field. In particular, the 3D surface scan has been acquired in an upright position whereas the generalized breast model was trained on prone MRI data as per the training set TS(B). Instead, the personalization of the generic breast model g(B) shape parameters can be derived by correlation with measurements collected from the instant torso T such the pectoral-muscle-to-mammilla-distance based on an x-ray mammogram or the composition of the breast tissue determined from a breast density estimate which can be likewise measured from the x-ray mammogram. In addition or instead, the correlation may be based on meta-data in relation to the instant torso, such as BMI, age, menopausal status, etc. Once the shape parameters have been determined through correlation, these can be passed on to a mesh generator (CAD component) to effect rendering of an adapted mesh model of shape m(B) which can then be used as input for the composite model generator CMG.

Alternatively, a biomechanical simulation is run on the generic shape g(B) to align same with the orientation as per the surface scan, so that both are now in the same orientation with respect to the gravitational field. One approach for such a re-orientation transformation envisaged herein is described in B Eiben et al in “Biomechanically guided prone-to-supine image registration of breast MRI using an estimated reference state”, published in conference proceedings, to “International Symposium on Biomedical Imaging (ISBI), 2013 IEEE 10th”, San Francisco, Calif., USA, 7-11 Apr. 2013, IEEE, 2013.

In case a machine learning algorithm has been trained, the personalization is particularly easy to achieve. The meta-data or measurements (collectively called md) are passed through the configured machine learning algorithm to obtain the shape parameter s and these are then used (as mentioned above) to CAD-render a corresponding mesh.

As can be seen, in either case the personalization by personalizer PS can be achieved for a given patient (torso T) without (direct) use of MRI data.

It will be understood that the above mentioned personalization embodiments can be simplified if applied to other parts of the human anatomy where the generic models and the available surface scan have all the same orientation relative to the gravitational field.

The personalization tool PS may be integrated into the composite model generator as a pre-processor or may be used as a separate, stand-alone tool altogether. In another case the personalizer encodes the respective functional relationships for the respective generic models and includes, preferably, input means to supply the meta-data and/or image information such as x-ray, mammography and others to carry out the personalization. Suitable user interface tools graphical, numerical or otherwise can be used. As mentioned earlier the meta-data can also be automatically fetched by suitably programmed interfaces that interface with database systems of a hospital information system, etc.

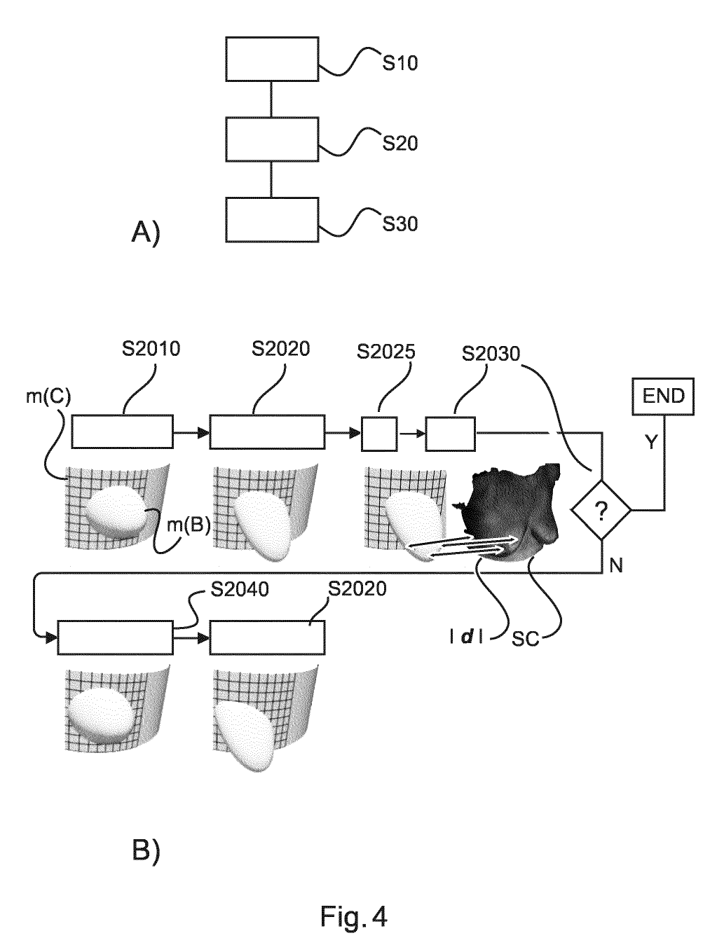

Reference is now made to the flow chart in FIG. 4A, where steps of a computerized composite model generation method are now described. The method steps provide further details on the operation of the generator CMG. However, it will be understood by those skilled in the art that the following description of the method can also be read in isolation and is not necessarily tied to the architecture described in FIG. 1.

At step S10 the two input models, in one embodiment that of the breast m(B) and the corresponding chest wall m(C), are received as input. Geometrically, the breast model m(B) is a mesh in the shape of an ellipsoidal cap and the chest wall model m(C) is roughly the lateral surface of a cylinder of elliptic cross section. In addition thereto the surface scan SC of the torso T is received (not necessarily at the same time and at the same input port).

In step S20 the two input models MB and MC are combined into a composite or combined model m(T) of the torso T.

At step S30 the so combined model m(T) is then stored or otherwise processed. For instance, the combined torso model MT may be used for a bio-medical simulation and this is graphically represented on a display device MT.

As explained earlier in FIG. 3, in one embodiment, the two input models have been obtained in separate learning procedures from respective generic models for the two anatomical components B, C. The respective training sets for the two models may include in one embodiment MRI imagery or obtained from other suitable soft-tissue imaging modalities.

With reference to FIG. 4B, the combination step S20 is now explained in more detail.

In an initial step S2010 the breast model m(B) is joined to the chest model m(C) at an initial position. In particular a control point of the model m(B) is set at said initial position on a surface of the chest model m(C). This can be done manually by the user in an interactive graphics environment where both input models m(B),m(C) are represented graphically in a common coordinate frame. The user uses a pointer tool (eg, computer mouse or other) to shift one of the models towards the other to effect the joining operation. A rotation may also be necessary to ensure that the longitudinal axis of the breast model m(B) is perpendicular to the longitudinal axis of the chest model cylinder m(C). In other embodiments, the joining operation S2010 is effected automatically. This can be achieved in one embodiment by detecting a characteristic part whose orientation/position is a tell-tale for that of the model. For instance, in an initial step the breast model can automatically be positioned by detecting the mammilla position in the optical surface scan and aligning it with the mammilla representation in the model m(B).

Broadly, the combination step, as proposed herein, includes in one embodiment an iterative optimization procedure and requires a set of initial optimization parameters. In one embodiment, the parameters to be optimized are the spatial components x, y and z of the control point. In one embodiment the control point is the breast center but other embodiments are also envisaged. In particular, as in this embodiment, only a single control point is estimated in the proposed method so as to correctly spatially locate the breast m(B) on the chest wall m(C). In another embodiment, a different optimization parameter other than the breast center may be used and/or a plurality of control points may be used for optimization. Initially, the control point co-ordinates are populated with initial values. The breast center may be taken to be the volumetric center of the breast mesh m(B) or, preferably, it is defined as the center-point of the elliptic base surface of the semi-ellipsoid/ellipsoidal cap breast model m(B).

Step S2010 essentially amounts to merging the two input models m(B) and m(C) into a single mesh model. While this merged or joined model is technically a single mesh, a labeling is performed in in one embodiment. In other words, descriptor labels are associated with some or all of the mesh elements in the joined up model to identify the structures, e.g. left breast surface. The labelling of the models can be used with benefit, to achieve a good initial guess as the breast center is assumed to lie on the posterior boundary of the breast model, that is, on the pectoral muscle/chest wall boundary.

The result of the joining operation at step S2010 constitutes an initial guess for the final combined torso model to be generated. In other words, this initial “candidate” combined model m(T)^{j=0} is in the following steps refined in one or more iterations to so arrive at the final combined torso model m(T)^{final}.

The combined model generation continues to step S2020, at which a bio-mechanical simulation is run based on the candidate combined model m(T)^{j}. At the conclusion of the bio-medical simulation a first configuration of the candidate bio-mechanical model m(T)^{j} is generated. The earlier mentioned TLED simulation package or others may be used. The biomechanical simulations are preferably run on a GPU (graphics processing unit) to further increase responsiveness.

In one embodiment, the simulation is configured to account for the earlier mentioned situation where one (or both) of the input models and the surface image have different orientations in relation to an applicable force field, such as the field of gravity. In other words, the candidate combined model m(T)^{j} is transformed into a configuration that corresponds to the same orientation relative to gravitational field at which the surface image has been recorded. More particularly, in one embodiment at least one, preferably both of the breast model m(T) and the chest wall model m(T) represents the geometry in a supine or prone position. Equally then the candidate combined model m(T)^{j} is likewise represented in supine or prone position whereas the surface scan SC has been recorded in upright position of the patient. In this situation, the simulation step S2020 allows transforming the configuration of m(T)^{j} into one an upright position as per the surface scan. In one embodiment of step S20, an approach is chosen similar to the one reported in the Eiben et al reference mentioned above. In Eiben's approach, both input models are first transferred from a loaded state into an “unloaded state” which represents a zero gravity environment. This is achieved by essentially reversing the gravity field components. Once a zero gravity configuration is achieved for both input models (treated together as the candidate combined model m(T)^{j}), a new loaded state is simulated by effecting an upright orientation in relation to the gravitational field. Alternatively and “dual” to this approach, it may be possible to transform the upright scan into prone or supine position using a similar approach. After the simulation, the candidate model m(T)^{j} is now “gravitationally” in alignment with the surface image SC. The simulation step S2020 serves as a quality check on whether the candidate combined models m(T)^{j} generated during the iterations actual converge towards the surface scan SC. In other words, the (one or more) simulations in step S2020 are preferably still done even if scan and the models already have the same orientation relative to the gravity field.

In step S2030 it is then determined an amount by which the surface scan SC and the candidate combined model m(T)^{j} deviate. A suitable deviation measure or cost function is defined for this. The cost function can be as simple as the square of the Euclidean surface distance |d|² between the two breast centers as per the surface scan and the candidate combined model m(T)^{j} or some other function of |d|², possibly with different weights αⱼ (eg, α₁(Δx)²+α₂(Δy)²+α₃(Δz)²) for the spatial coordinates x,y,z. Other cost functions are also envisaged. If the cost function is found to return a value below a pre-defined threshold the method terminates and the currently stored candidate model is output as the final combined torso model m(T)^{final}. The distance between the the candidate combined model m(T)^{j} the the surface scan SC can be computed using the assigned structure labels from the labelling and the fact that the surfaces are aligned (registered) to each other in a common coordinate system (registration will be explained in more detail further below at step S2025).

If however the deviation is found to exceed a pre-defined threshold, the method continues to step S2040 where a new, second, follow-up candidate combined model m(T)^{j+1} is generated. This is achieved in one embodiment by varying the initial attachment position originally set at step S2010.

Once a new attachment position has been set, the method flow now returns to step S2010 by re-attaching the initial breast model to the chest wall model at the different position determined in optimization step S2040. The method now enters the follow up iteration step iteration j+1. The bio-mechanical simulation at step S2020 is then re-run based on the new follow-up candidate combined model m(T)^{j+1} which defines a new configuration of the earlier candidate model m(T)^{j} by virtue of the change of the attachment point as determined in step S2040.

The proposed method can thus be seen to be implementable in a double loop scheme where the outer loop is formed by step S2030 by repeatedly comparing the cost function whilst the inner loop is formed by step S2040 in which the attachment point between breast and chest wall is being varied according to an optimization scheme. A suitable optimization scheme underlying operation of the optimization step S2040 as explained above in relation to FIG. 4B includes local optimizers of the likes of Nelder-Mead, conjugate gradients, Newton-Raphson or others, or more global approaches such as differential evolution, controlled random search, or others.

The optimization loop at step S2040 may be implemented as follows in one embodiment: the elements (eg, surface triangles) of the breast portion in the model m(T)^{j} are varied in one more DoFs, eg are translated, to a new candidate positions relative to the initial center point on the posterior boundary. The (new) center breast distance in this new configuration is then evaluated by using the cost function. The new breast center whose position minimizes the distance to the surface mesh is used as a new breast center position for model composition at step S2010. The position of the breast center point of breast model m(B) is varied relative to the chest model m(C) and the models are then recomposed at step S2010, and this is repeated throughout the iterations. The new breast center is thus the new attachment point when the iteration returns to step S2010. As will be appreciated, in this embodiment the control point and the attachment point are the same (the breast center that is), but this may not be so in other embodiments/anatomies where control point and attachment point differ.

The degrees of freedom over which the simulation at step S2020 is run may differ through the course of the iterations. For instance, a larger set of mechanical degrees of freedom such as rotation, translation and deformation etc are admitted in the first iteration given the initial guess for the attachment point at which the two sub-models m(B) and m(T) are initially attached to each other. However, in order to save CPU time in subsequent iterations when re-visiting the bio-mechanical simulation to test the correspondence between the candidate model and the surface scan, only a restricted set of degrees of mechanical freedom are allowed to simplify the bio-mechanical simulation. In one embodiment the allowable degrees of freedoms are for instance mere translations. In other embodiments only rotations are allowed in subsequent or later iterations at the bio-mechanical simulations S2020. In addition or instead, the DoFs for varying the attachment point/control point (originally set at step S2010) in the optimization step S2040 may also be confined to certain hyperplanes for instance or other dynamic restrictions are imposed. For instance, only variation in a predefined boundary of mesh elements with specified labelling are allowed to thereby ensure that prior anatomical knowledge is respected. Again, confining attachment point variability in this manner helps reduce CPU time. This anatomical knowledge conferred by the labelling can also be harnessed in a similar manner in step S2040 when searching for the optimal distance between the breast center points as per model m(T)^{j} and surface scan SC.

The optimization in step S2040 may not necessarily return a global minimum but only local one. Also, the out final composite model m(T)^{FINAL}may not even be a local minimum as one may, in some embodiments, simply abort iterations once the cost function returns a value below a pre-defined quality threshold or once follow-up candidate models generated during the iterations differ by less than a pre-defined threshold from one another. A single iteration step may suffice but the method may in general require a plurality of iterations.

Although the above has been explained in relation to a minimization problem where the cost function is to be minimized this is not limiting as in other context a reformulation in terms of a maximization problem (where utility function is to be maximized) may be more befitting and these variants are particularly likewise envisaged herein.

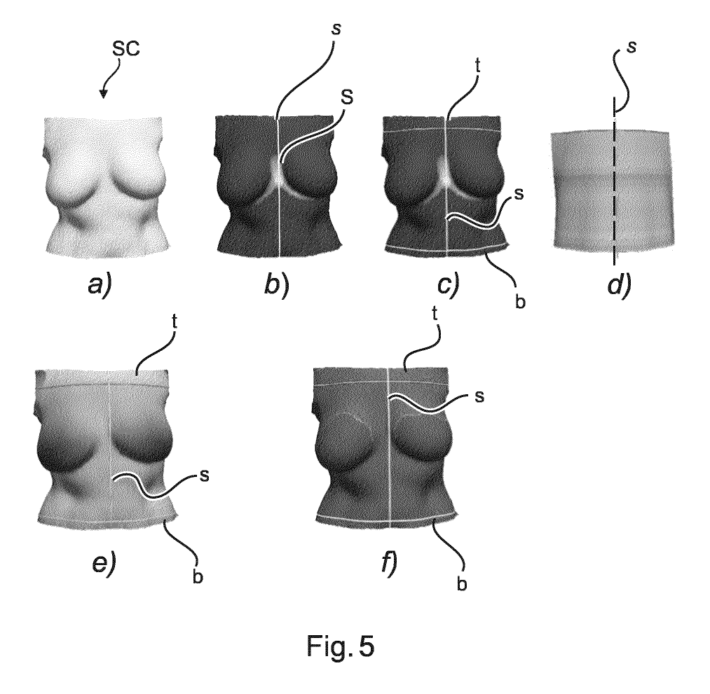

In order to define the cost function, in particular, in order to compute the surface distance between the candidate model m(T)^{j} and the surface scan SC at any given stage in the iteration, a registration step S2025 is required which will now be described in more detail with reference to the illustrations in FIG. 5.

In one embodiment the geometrical registration can be achieved by simplifying the surface scan into a simpler geometrical structure that roughly represents the geometry of the chest model. In particular, registration is carried out only in respect to the chest model. In particular, the candidate combined model as whole is not used for registration in this embodiment, although this can still be done in other embodiments if desired.

More specifically, a suitable surface mesh for the chest wall m(C) has been found to be cylinders with elliptical cross section. The task is then to simplify the surface scan into such a cylinder. To achieve the simplification of the surface scan in a first step the breast portions are virtually removed from the upright scan SC. A new surface mesh without the breast mesh element (eg triangles) is then generated and this is then fitted to a cylinder to so approximate the torso shape. Translation, rotation and eccentricity of the simple shape are optimized and standard optimization algorithm can be used for this. The so simplified surface scan and chest model mC can then be registered by aligning the principle axes of the respective elliptical cross sections. This then induces automatically a registration between the original scan SC and the mode mC.

The step of removing the breast portions from the surface scan to achieve the simplified cylindrical mesh can be implemented in a number of different ways, one of which will be described in more detail in the following, with the understanding that this is not necessarily limiting.

In this approach, a control point on the surface scan is identified that roughly represents a surface point of the simplified cylinder one wishes to construct. A system of lines is then defined in relation to this control point. The lines are then used to propagate the control point around at least a part of the surface scan to thereby “shear off” excess volume so as to define the simplified cylindrical shape.

Initially, appropriate sub-portions of the 3D surface scanner are selected (eg, full width without arms approximately from jugular notch to navel). This is illustrated in FIG. 5a).

In one embodiment, the sternum S is used as the control point and this can be found by using curvature and position information. The system of lines earlier mentioned are then defined based in one embodiment as a system of three lines (it should be noted that the notion of “lines” in the following are lines on curved surfaces): one line s is run through the sternum S from top to bottom as illustrated in FIG. 5b).Another line b is run at about 10% height, defined by intersection with a plane parallel to the floor as illustrated in FIG. 5c). The third Line t is run at about 90% height, defined by intersection with a plane parallel to the floor as illustrated in FIG. 5c). The “top” and “bottom” lines t and b are resampled to the same number of points. These lines, together with sternum line s, allow defining a local coordinate system. In the following, a specific point on any of the three lines is parameterized along the respective line by multiplication with a scalar i ∈[0,1]: for i=0, the parameterized point is at one end of the respective line segment, whereas for i=1 the parameterized point is at the other end. The propagation of the sternum S is effected by varying parameter i. An auxiliary surface A is then generated by the following sub-routine (as illustrated in FIG. 5d): both lines b and t are aligned by matching their intersections with line S. For each point on line b, a translation vector to the aligned point in line t is computed as (t−b). Starting from, say, line b, additional lines are generated by applying the following transformation along line S and interpolating between the two line shapes: i*s+i*(t−b).

Next, a distance from the surface SC to the surface A is computed as illustrated by the shading in FIG. 5e). A feature vector for each point in scan SC is generated using the computed distance and curvature at the respective point/mesh element. K-means clustering with 3 classes (or other quantization or clustering technique) is used to identify points belonging to the breast portion as illustrated in FIG. 5f) where the breast portions are now isolated from the remaining torso illustrated by the shading. These “breast mesh elements” (rendered in dark shading in FIG. 5f) are then removed and the desired new surface N mesh without breast triangles is obtained.

Although envisaged herein as the preferred embodiment, it will be understood that the human female torso is merely one embodiment of the biomechanical assembly. That is, the proposed imaging processing system may also be applied with benefit to other parts of the human (or animal) anatomy.

In another exemplary embodiment of the present invention, a computer program or a computer program element is provided that is characterized by being adapted to execute the method steps of the method according to one of the preceding embodiments, on an appropriate system.

The computer program element might therefore be stored on a computer unit, which might also be part of an embodiment of the present invention. This computing unit may be adapted to perform or induce a performing of the steps of the method described above. Moreover, it may be adapted to operate the components of the above-described apparatus. The computing unit can be adapted to operate automatically and/or to execute the orders of a user. A computer program may be loaded into a working memory of a data processor. The data processor may thus be equipped to carry out the method of the invention.

This exemplary embodiment of the invention covers both, a computer program that right from the beginning uses the invention and a computer program that by means of an up-date turns an existing program into a program that uses the invention.

Further on, the computer program element might be able to provide all necessary steps to fulfill the procedure of an exemplary embodiment of the method as described above.

According to a further exemplary embodiment of the present invention, a computer readable medium, such as a CD-ROM, is presented wherein the computer readable medium has a computer program element stored on it which computer program element is described by the preceding section.

A computer program may be stored and/or distributed on a suitable medium (in particular, but not necessarily, a non-transitory medium), such as an optical storage medium or a solid-state medium supplied together with or as part of other hardware, but may also be distributed in other forms, such as via the internet or other wired or wireless telecommunication systems.

However, the computer program may also be presented over a network like the World Wide Web and can be downloaded into the working memory of a data processor from such a network. According to a further exemplary embodiment of the present invention, a medium for making a computer program element available for downloading is provided, which computer program element is arranged to perform a method according to one of the previously described embodiments of the invention.

It has to be noted that embodiments of the invention are described with reference to different subject matters. In particular, some embodiments are described with reference to method type claims whereas other embodiments are described with reference to the device type claims. However, a person skilled in the art will gather from the above and the following description that, unless otherwise notified, in addition to any combination of features belonging to one type of subject matter also any combination between features relating to different subject matters is considered to be disclosed with this application. However, all features can be combined providing synergetic effects that are more than the simple summation of the features.

While the invention has been illustrated and described in detail in the drawings and foregoing description, such illustration and description are to be considered illustrative or exemplary and not restrictive. The invention is not limited to the disclosed embodiments. Other variations to the disclosed embodiments can be understood and effected by those skilled in the art in practicing a claimed invention, from a study of the drawings, the disclosure, and the dependent claims.

In the claims, the word “comprising” does not exclude other elements or steps, and the indefinite article “a” or “an” does not exclude a plurality. A single processor or other unit may fulfill the functions of several items re-cited in the claims. The mere fact that certain measures are re-cited in mutually different dependent claims does not indicate that a combination of these measures cannot be used to advantage. Any reference signs in the claims should not be construed as limiting the scope.

1. System of generating a composite model for a bio-mechanical assembly, comprising:
an input interface (IN) for receiving i) at least two input component models (m(B), m(C)) for respective anatomical components (B,C) of the mechanical assembly, and ii) a surface image acquired by a camera (DSC) of an outer layer (OL) of said biomechanical assembly (T); and
a combiner (Σ) configured to combine, based on said surface image, said at least two input component models (m(B), m(C)) into a combined mechanical model (m(T)) for said biomechanical assembly.

2. System of claim 1, with at least one of said at least two input component models (m(B), m(C)) previously adapted from a generic model (g(B), g(C)) for a respective one of the anatomical components.

3. System of claim 1, wherein the at least two input models (m(B), m(C)) have been so adapted from respective generic models (g(B), g(C)), with said generic models (g(B), g(C)) separately learned from respective, different, training sets.

4. System of claim 1, wherein the combiner includes a solver (SLV) component configured to:
join a first one (m(B)) of the input component models to a second one (m(C)) of the input component models at an initial position of said second input component model (m(C)), to so obtain a candidate combined mechanical model;
perform a mechanical simulation of the candidate combined model to obtain a first configuration of the candidate combined model (m(T));
compare said configuration of the candidate combined model with the surface image to obtain a measure of deviation; and
based on said measure of deviation, to vary said initial position to obtain a second candidate combined model (m(T)ⁱ⁺¹).

5. System of claim 4, where the bio-mechanical simulation is performed over a plurality of mechanical degrees of freedom.

6. System of claim 5, wherein said varying of said initial position is performed only for a subset of said plurality of said mechanical degrees of freedom.

7. System of claim 5, wherein the solver (SLV) proceeds iteratively in iteration steps through a series of candidate combined models, wherein a number of the mechanical degrees of freedom varies with said iteration steps.

8. System of 1, wherein the bio-mechanical assembly (T) is an animal or human torso.

9. System of claim 1, wherein the anatomical components include a) a breast and b) a chest wall.

10. A computer-implemented method of generating a composite model for a biomechanical assembly (T), comprising the steps of:
receiving (S10) i) at least two input component models (m(B), m(C)) for respective anatomical components (B,C) of the mechanical assembly, and ii) a surface image acquired by a camera (DSC) of an outer layer (OL) of said biomechanical assembly (T); and
based on said surface image, combining (S20) said at least two input component models (m(B), m(C)) into a combined mechanical model (m(T)) for said biomechanical assembly.

11. Method of claim 10, with at least one of said at least two input component models (m(B), m(C)) previously adapted from a generic model (g(B), g(C)) for a respective one of the anatomical components.

12. Method of claim 10, wherein the at least two input models (m(B), m(C)) have been so adapted from respective generic models (g(B), g(C)), with said generic models (g(B), g(C)) separately learned from respective, different, training sets.

13. Method of claim 10, wherein the combining step (S20), comprises:
from the at least two input component models, joining (S2010) a first one (m(B)) of the input component models to a second one (m(C)) of the input component model at an initial position of said second input component model (m(C)), to so obtain a candidate combined mechanical model;
performing (S2020) a mechanical simulation of the candidate combined model to obtain a first configuration of the candidate combined model (m(T)ⁱ);
comparing (S2030) said configuration of the candidate combined model with the surface image to obtain a measure of deviation; and
based on said measure of deviation, varying (S2040) said initial position to obtain a second candidate combined model (m(T)^{i+l}).

14. (canceled)

15. (canceled)
